(19) United States
(12) Patent Application Publication
(10) Pub. No.: US 2016/0044747 A1
(43) Pub. Date: Feb. 11, 2016

(54) MODULAR ANTI-FOG DEVICES

(71) Applicants: Lincoln Dale Prins, Cedar Rapids, IA (US); James Anthony Mapes, Lakewood, WA (US)

(72) Inventors: Lincoln Dale Prins, Cedar Rapids, IA (US); James Anthony Mapes, Lakewood, WA (US)

(21) Appl. No.: 14/821,510

(22) Filed: Aug. 7, 2015

Related U.S. Application Data
(60) Provisional application No. 62/035,194, filed on Aug. 8, 2014.

(51) Int. Cl. H05B 3/86; H05B 3/06; A61F 9/02

(52) U.S. Cl. CPC H05B 3/86; A61F 9/029; H05B 3/06

(57) ABSTRACT

A modular anti-fog device for attachment to a wearable optical device such as glasses, goggles, and the like. The anti-fog device includes a translucent carrier configured to hold a conductive element. The conductive element is electrically coupled to a power source. The conductive element provides resistive heating upon passage of electrical current through the conductive element. The translucent carrier includes an adhering layer enabling the anti-fog device to be attached to an optical device so as to provide the optical device with anti-fog functionality.

### CROSS-REFERENCE TO RELATED APPLICATIONS

This application is a non-provisional application claiming priority to and the benefit of U.S. Patent Application Ser. No. 62/035,194, filed on Aug. 8, 2014 and titled “MODULAR ANTI-FOG DEVICES,” the entirety of which is expressly incorporated herein by this reference.

### BACKGROUND

The present disclosure relates generally to anti-fog devices useful for reducing or preventing fogging of a viewing portion of a wearable optical device, such as goggles, glasses, helmets, face shields, visors, masks, etc.

A continuing problem related with the use of optical devices in industrial, medical, military, sports, and home-use applications is visual impairment caused by fogging of the viewing portion of the optical devices. Typically, warm and moist air exhaled by a wearer condenses on the relatively cooler surfaces of the optical device, resulting in condensation and a “fogging” of the viewing surface. Such fogging and concomitant visual impairment can lead a user to remove the wearable optical devices and therefore abandon the intended benefits of the device and/or can lead to an increase in accident risk.

It is known that embedding conductive wires within the substrate of a viewing portion of an optical device and electrically coupling the conductive wires to a power source to provide current to the conductive wires can heat the conductive wires to bring the viewing portion to a temperature above the condensation temperature of moisture in the nearby air. Typically, optical devices providing such a function are formed as specialty items having the power source and conductive wires embedded into the design and structure of the device. A user is thus limited to the styles, sizes, and applications to which the specialty item is specifically designed.

### BRIEF SUMMARY

The present disclosure is related to modular anti-fog devices and systems configured to provide a standard wearable optical device (e.g., an optical device not having anti-fog functionality) with anti-fog functionality.

One or more embodiments of the present disclosure relate to a modular anti-fog device, including a translucent carrier formed of a translucent material, the translucent carrier having a first side and a second side, the first side having an adhering surface; a conductive element formed into a pattern that is positioned within or against the translucent carrier, the conductive element configured to provide heat as a result of the passage of electric current through the conductive element; and a power supply electrically coupled to the conductive element and configured to provide the conductive element with electric current; wherein the translucent carrier is configured to be attachable to a wearable optical device by positioning the adhering surface of the translucent carrier against at least a portion of the wearable optical device, thereby providing the wearable optical device with anti-fog functionality or enhanced anti-fog functionality.

One or more embodiments of the present disclosure relate to a wearable optical device configured with anti-fog functionality, including: an anti-fog device, the anti-fog device including a translucent carrier formed of a translucent material, the translucent carrier having a first side and a second side, the first side having an adhering surface, a conductive element formed into a pattern that is positioned within or against the translucent carrier, the conductive element configured to provide heat as a result of the passage of electric current through the conductive element, and a power supply electrically coupled to the conductive element and configured to provide the conductive element with electric current; and a wearable optical device, wherein the translucent carrier is configured to be attachable to the wearable optical device by positioning the adhering surface of the translucent carrier against at least a portion of the wearable optical device, thereby providing the wearable optical device with anti-fog functionality or enhanced anti-fog functionality.

One or more embodiments of the present disclosure relate to an anti-fog kit configured to provide a standard wearable optical device with anti-fog functionality, the kit including: an anti-fog device having a translucent carrier formed of a translucent material, the translucent carrier having a first side and a second side, the first side having an adhering surface, a conductive element formed into a pattern that is positioned within or against the translucent carrier, the conductive element configured to provide heat as a result of the passage of electric current through the conductive element, and a power supply electrically coupled to the conductive element and configured to provide the conductive element with electric current; an adhesive configured to be applied to the adhering surface of the anti-fog device; and a wearable optical device, wherein the translucent carrier is configured to be attachable to the wearable optical device by positioning the adhering surface of the translucent carrier against at least a portion of the wearable optical device, thereby providing the wearable optical device with anti-fog functionality or enhanced anti-fog functionality.

### BRIEF DESCRIPTION OF THE DRAWINGS

To further clarify the above and other advantages and features of the present disclosure, a more particular description of the disclosure will be rendered by reference to specific embodiments thereof which are illustrated in the appended drawings. It is appreciated that these drawings depict only illustrated embodiments of the disclosure and are therefore not to be considered limiting of its scope. Embodiments of the disclosure will be described and explained with additional specificity and detail through the use of the accompanying drawings in which:

FIG. 1 illustrates an embodiment of an anti-fog device according to the present disclosure;

FIG. 2 illustrates an embodiment of an anti-fog device applied to an optical device according to the present disclosure;

FIG. 3 illustrates another embodiment of an anti-fog device according to the present disclosure;

FIG. 4 illustrates another embodiment of an anti-fog device applied to an optical device according to the present disclosure;

FIG. 5 illustrates a cross-sectional view of an anti-fog device according to the present disclosure showing a plurality of layers of the anti-fog device; and

FIG. 6 illustrates another embodiment of an anti-fog device according to the present disclosure including thermal contacts.

### DETAILED DESCRIPTION

The present disclosure relates to anti-fog devices useful for reducing or preventing fogging of a viewing portion of a wearable optical device, such as goggles, glasses, helmets, face shields, visors, masks, etc. Some embodiments may be useful for reducing or preventing fogging of a viewing portion of a non-wearable optical device, such as a scope, sight, binoculars, etc. Although the disclosure may particularly reference “wearable optical devices” in relation to embodiments of anti-fog devices, it will be appreciated that one or more of such embodiments may be useful in applications involving non-wearable optical devices as well.

As used herein, the term “wearable optical device” and similar terms refer to goggles (e.g., dive, ski), glasses, helmets, face shields, visors, masks, screens, lenses, ballistic eyewear, athletic eyewear, and other eye protection gear and headgear that may be worn by a user to provide protection (e.g., from solar radiation, intense light, and/or foreign objects contacting the eyes, face, and/or head), comfort, vision enhancement (e.g., glare reduction), and/or for other reasons according to user needs and preferences.

As used herein, the terms “conductive element,” “conductive material,” and similar terms refer to conductive materials having an electrically conductive component and/or a thermally conductive component. For example, a conductive element can have an electrically conductive component that enables the conductive element to resistively conduct electricity so as to generate heat as electrical current is passed through the conductive element.

In some embodiments, the thermally conductive component may enable the conductive element to receive heat transmitted from other portions of the anti-fog device, from distal areas of a connected wearable optical device, and/or from a user of the wearable optical device. In some embodiments, the thermally conductive component will overlap with the electrically conductive component such that both components can be exhibited by the same material or combination of material(s) (e.g., copper wiring having suitable electrical conductivity and thermal conductivity). In other embodiments, the thermally conductive component can be separate from the electrically conductive component. For example, some embodiments may include an electrically conductive component primarily configured to provide resistive heating upon the passage of electrical current, and a thermally conductive component primarily configured to provide heat transfer to and throughout the conductive element, though there may be overlap between the separate functions performed by each component.

Conductive elements can include metal wiring, conductive thermoplastic, conductive paint, and/or conductive ink. When the conductive material includes a wired configuration, it will be appreciated that the wiring can have a cross-sectional shape that is cylindrical or angular. In some embodiments, the conductive element can be formed from a brass material (e.g., a copper and zinc alloy) or from a copper material. In some embodiments, the conductive material can include a coating of nickel and/or cadmium (e.g., a brass or copper material coated in a nickel-cadmium alloy). In other embodiments, the conductive element may be formed from other materials (e.g., other metals) capable of producing sufficient heat upon passage of electrical current through the material and/or capable of sufficient thermal conductivity. In some embodiments, for example, the thermally conductive component can include aluminum, copper, diamond, gold, molybdenum, tungsten, aluminum nitride, beryllium oxide, silicon carbine, and/or other thermally conductive metals or compounds.

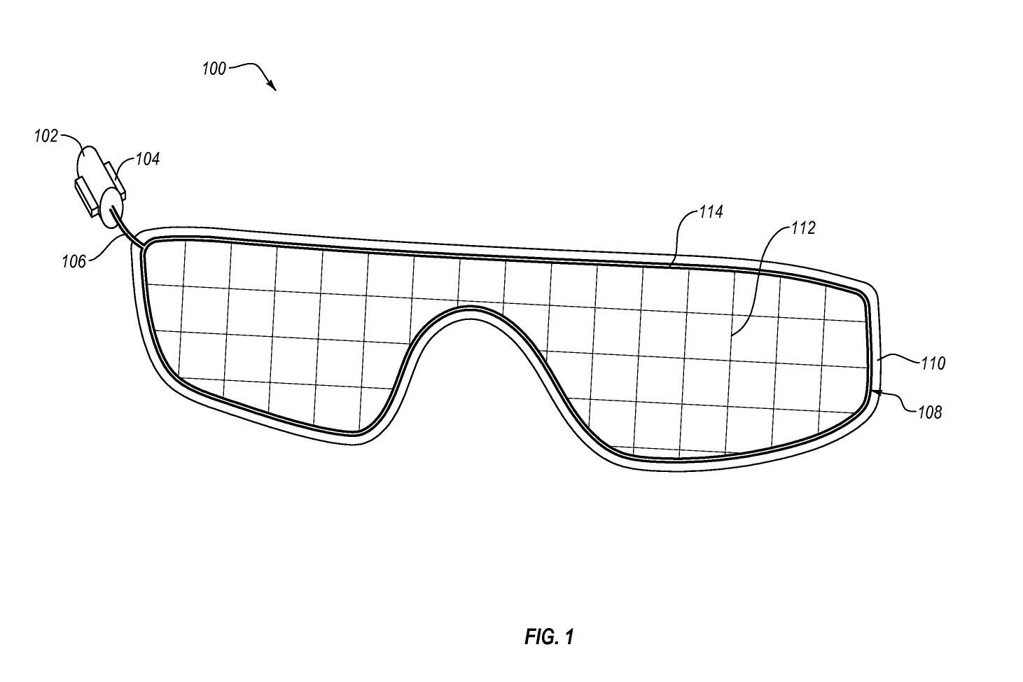

FIG. 1 illustrates an embodiment of an anti-fog device 100 configured to be attachable to a wearable optical device to provide anti-fogging functionality to the wearable optical device. As illustrated, the anti-fog device 100 can include a power supply 102 electrically coupled to a conductive element 108. As shown, the conductive element 108 can be formed from conductive wiring configured in a series of lines, a grid, or other pattern. In some embodiments, the conductive element 108 includes thin or small gauge metal wiring having a resistance configured to provide, when current is passed through, sufficient heat to raise the nearby temperature to above the moisture condensation temperature when current is passed through the metal wiring.

In some embodiments, the conductive element 108 can include portions of varying size and/or type of conductive wiring. For example, the conductive element 108 can include one or more portions of lower resistance (e.g., as a result of wider diameter wiring and/or being formed of a lower resistance material) and one or more portions of higher resistance (e.g., as a result of smaller diameter wiring and/or being formed of a higher resistance material). As illustrated, the conductive element 108 can include a peripheral portion 114 formed from a wider or larger diameter wiring and an inner portion 112 formed from a narrower or smaller diameter wiring. In some embodiments, the conductive element 108 can be configured such that the peripheral portion provides less electrical resistance than the inner portion 112 (e.g., as a result of the peripheral portion 114 having a larger diameter or being wider).

Such embodiments can beneficially provide heat generation that is relatively greater at the inner portion 112 and areas adjacent to the inner portion 112 as compared to heat generation at the peripheral portion 114 and areas adjacent to the peripheral portion 114. For example, the inner portion 112 can be configured to cover areas of an optical device more likely to fog and/or areas more directly associated with a user's field of vision, thereby providing greater heating, faster de-fogging, and/or greater fogging prevention. In addition, the peripheral portion 114 can be configured to cover areas farther from a user's field of vision and/or less likely to fog. The peripheral portion 114 can be configured such that the lower resistance of the peripheral portion 114 provides sufficient heating for associated portions of an optical device to which it is attached while at the same time minimizing resistance such that a greater portion of an applied voltage can be utilized by the wiring of the inner portion 112. For example, in embodiments having a power supply 102 configured as a direct-current source (e.g., battery, thermocouple, and/or solar cell), a greater portion of the supplied voltage can be applied to the inner portion 112, and the voltage drops of the circuit can be greater across sections of the inner portion 112. One or more embodiments of anti-fog devices of the present disclosure, such as anti-fog device 100, can thus be configured to provide power efficient anti-fogging functionality to an attached optical device.

As illustrated, the conductive element 108 can be configured to have a grid configuration. In other embodiments, the conductive element 108 can be configured as a series of parallel lines (e.g., horizontal, vertical, or angled), or as a series of non-parallel lines, curving lines, spiral lines, and/or any other pattern that is adequate to cover desired portions of a wearable optical device upon application of an anti-fog device to the wearable optical device.

The conductive element 108 can have wiring having a diameter ranging from about 0.01 to about 1 mm, or from about 0.025 to about 0.25 mm, or from about 0.05 to about 0.1 mm.

As illustrated, the optical device 100 can include a translucent carrier 110 configured to hold the conductive element 108 in a desired configuration. For example, the translucent carrier 110 can function as a substrate upon which the conductive element 108 is fixedly attached so as to preserve the orientation of the various components of the conductive element 108. In some embodiments, the conductive element 108 may be embedded within the translucent carrier 110. In other embodiments, the conductive element 108 may be biased against a single side of the translucent carrier 110.

In addition, the translucent carrier 110 can be configured to be attachable to a viewing portion (e.g., lens, screen, window, glass) of an optical device. For example, the translucent carrier 110 can include an adhesive surface allowing the anti-fog device 100 to be placed against the viewing portion of an optical device. In some embodiments, an adhesive surface of the translucent carrier 110 can also provide for attachment of the conductive element 108 to the translucent carrier 110. For example, the conductive element 108 may be adhered to the translucent carrier 110 on one side of the translucent carrier 110, and that same side can be applied to the viewing portion of an optical device. In other embodiments, the translucent carrier 110 omits an adhesive, and may be configured to bias against a viewing portion of an optical device through static forces (e.g., electrostatic attraction).

The translucent carrier 110 can be formed from vinyl, polyurethane, polyethylene terephthalate (PET), other polymeric materials, or combinations of such polymeric materials. In some embodiments, the translucent carrier 110 can be configured (e.g., through material selection and/or material thickness selection) as a flexible film providing sufficient flexibility to allow a user to fold, bend, or otherwise orient the anti-fog device 100 so as to match the shape of an optical device to which it is attached. In other embodiments, the translucent carrier 110 can be configured (e.g., through material selection and/or material thickness selection) to have a pre-configured shape. For example, the translucent carrier 110 may be configured to have a rigid or semi-rigid shape formed so as to match the shape of an optical device (or portion thereof) to which it may be applied.

In some embodiments, the translucent carrier 110 may be configured to provide one or more additional properties and/or optical adjustments. For example, the translucent carrier 110 can be formed of a scratch-resistant and/or shatter-resistant material. In some embodiments, the translucent carrier 110 can be configured to reduce glare and/or to polarize light passing through the translucent carrier 110. In some embodiments, the translucent carrier can be tinted, colored, or shaded to limit the type and/or amount of light passing through the translucent carrier 110. In some embodiments, the translucent carrier 110 can be configured to provide ultra-violet light resistance.

As illustrated, the power supply 102 can be electrically coupled to the conductive element 108 through one or more leads 106. The power supply 102 is configured to provide power to the conductive element 108 in order to generate heat for preventing and/or removing fog. In some embodiments, the power supply 102 can be configured as a battery cartridge for holding one or more batteries as a power source. Additionally, or alternatively, one or more solar cells, thermocouples, and/or kinetic energy harvesters (e.g., piezoelectric devices) may be utilized to provide power to the conductive element 108.

As shown in the illustrated embodiment, the power supply 102 can be coupled to an attachment 104 configured to allow the power supply 102 to be secured to an optical device. For example, the attachment 104 can be formed as a clip, as shown. Other embodiments can include one or more straps, pins, latches, hooks, clamps, loops, adhesives, hook and loop fasteners, and/or other fasteners allowing the power supply 102 to be secured to an optical device upon attachment of the anti-fog device 100 to the optical device.

The power supply 102 can also include a controller configured to cycle power on and off and/or to adjust power delivery to the conductive element 108. For example, power cycling may follow a pre-determined timing schedule, can be user controlled (e.g., actuated on demand by pressing a button, adjusting a dial, etc.), and/or can be dynamically modified in response to detected ambient conditions (e.g., humidity, temperature, translucency through a viewing portion of an optical device). Accordingly in some embodiments, the power supply 102 may include and/or may be in communication with one or more sensors, including temperature sensors, humidity sensors, light/translucence sensors and/or other sensors for detecting ambient conditions.

In some embodiments, the power supply 102 may have a power actuator (e.g., button, dial, slider, switch, or other actuator) which, when actuated, initiates a power cycle to pass power to the conductive element 108 for a predetermined period of time. This predetermined period of time can be less than a second, one second, a few seconds (e.g., 2-10 seconds), many seconds (e.g., 10-60 seconds), or several minutes (e.g. 2 or more minutes), or until the power button is pushed again to turn off the power. A light indicator can also be provided next to the power actuator to indicate when power is cycling through the conductive element 108.

The size and shape of the conductive element 108 can be varied. In embodiments including metal wiring, the wiring may have a cross-sectional shape that is flat, round, oval, polygonal, or otherwise shaped. The size and shape of the conductive element 108 can vary according to the conductivity of the material from which it is formed, the strength of the power supply 102, the frequency of power cycles and duration of the power cycles provided by the power supply 102, the type of translucent carrier 110 that the conductive material is applied to, as well as the size and shape of a wearable optical device to which the anti-fog device 100 is attached and ambient conditions in which the wearable optical device is used. The strength of the power supply and the cycles of the power supply through the conductive element 108 can also be varied to accommodate these variables, so as to provide a desired effect.

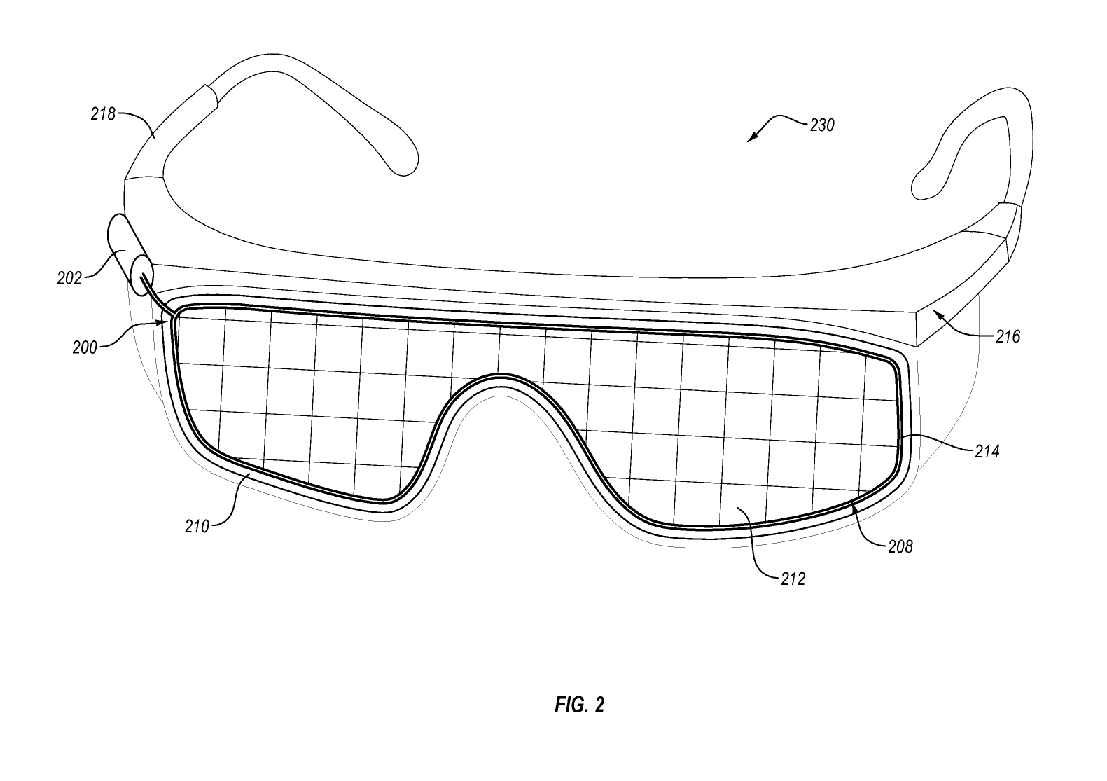

FIG. 2 illustrates an anti-fog system 200 including an anti-fog device 200 attached to a wearable optical device 216. As illustrated, a power supply 202 of the anti-fog device 200 can be attached to the wearable optical device 216 at a temple portion 218 (e.g., using a clip and/or other fastener as described above). As shown, the anti-fog device 200 can include a translucent carrier 210 that holds a conductive element 208 (which, in this embodiment, includes a peripheral portion 214 and an inner portion 212) and that allows the anti-fog device 200 to be placed against a viewing portion of the wearable optical device 216.

As illustrated, the size and shape of the anti-fog device 200 can be configured to substantially match the viewing portion of the wearable optical device 216 that the anti-fog device is placed upon, such as the glasses shape shown in the Figure. In alternative embodiments, the size and shape of an anti-fog device can be configured as a simple rectangle, oval, circle, triangle, polygon, or other desired shape.

As illustrated, the anti-fog device 200 can be configured to cover substantially the entire viewing portion of the wearable optical device 216. In other embodiments, an anti-fog device can be configured to cover a limited section of a viewing portion of a wearable optical device, such as a circular or rectangular area immediately in front of a position of a user's eyes, or a horizontal strip that crosses in front of a position of a user's eyes.

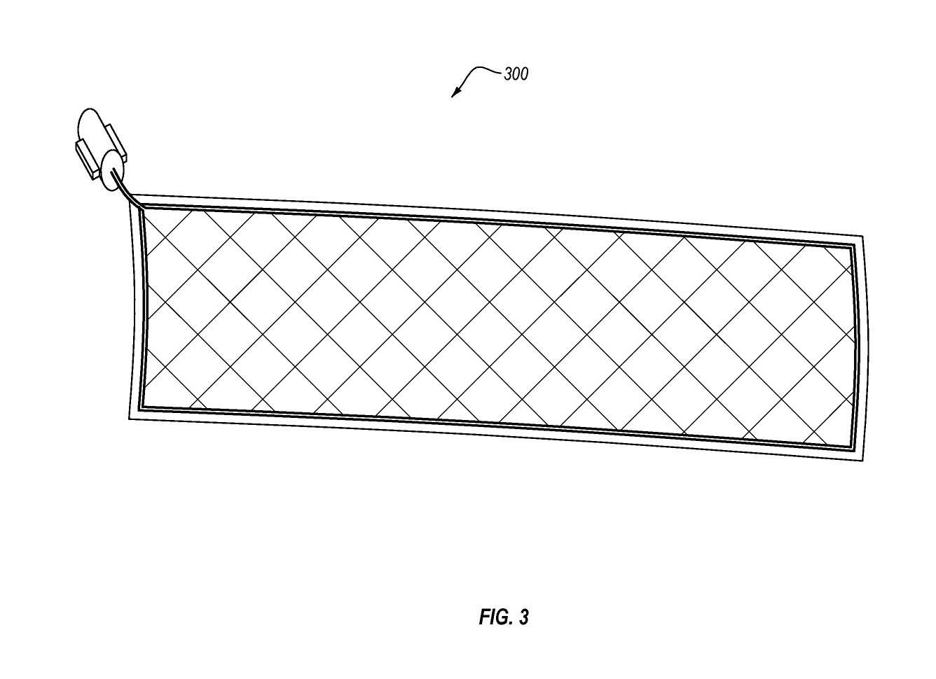

FIG. 3 illustrates another embodiment of an anti-fog device 300. As illustrated, the anti-fog device 300 can be formed in a substantially rectangular shape. Such an embodiment may be suitable, for example, for placement upon a visor, window, or other viewing portion of an industrial or medical helmet or face shield (e.g., welder's helmet, firefighter's helmet, surgeon's face shield). Such an embodiment may also be suitable for use with goggles, glasses, and/or other types of wearable optical devices described herein.

The illustrated embodiment, as well as other embodiments described herein, can be configured as modular devices that may be shaped and/or formed into a desired shape (e.g., a shape matching the shape of a desired wearable optical device). For example, the anti-fog device 300 may be cut, folded, bent, twisted, or otherwise shaped and/or oriented so as to fit the profile of a selected wearable optical device. The anti-fog device 300 can then be placed against the wearable optical device to provide the wearable optical device with the anti-fog functionality and benefits of the anti-fog device 300. Such a configuration can provide a number of advantages. For example, a user can select a preferred wearable optical device and can easily modify the device by coupling an anti-fog device upon it to provide anti-fog functionality. Accordingly, a user can select a wearable optical device having an appropriate fit, comfort, size, shape, field of view, and/or other features for a given application, and can equip the selected wearable optical device with anti-fog functionality, even if the selected wearable optical device is a standard optical device (e.g., not having built in anti-fog capabilities). As opposed to selecting from a limited number of specially designed and specific-purpose optical devices having embedded anti-fogging systems, a user can therefore have greater options and a greater number of applications in which anti-fog functionality is available.

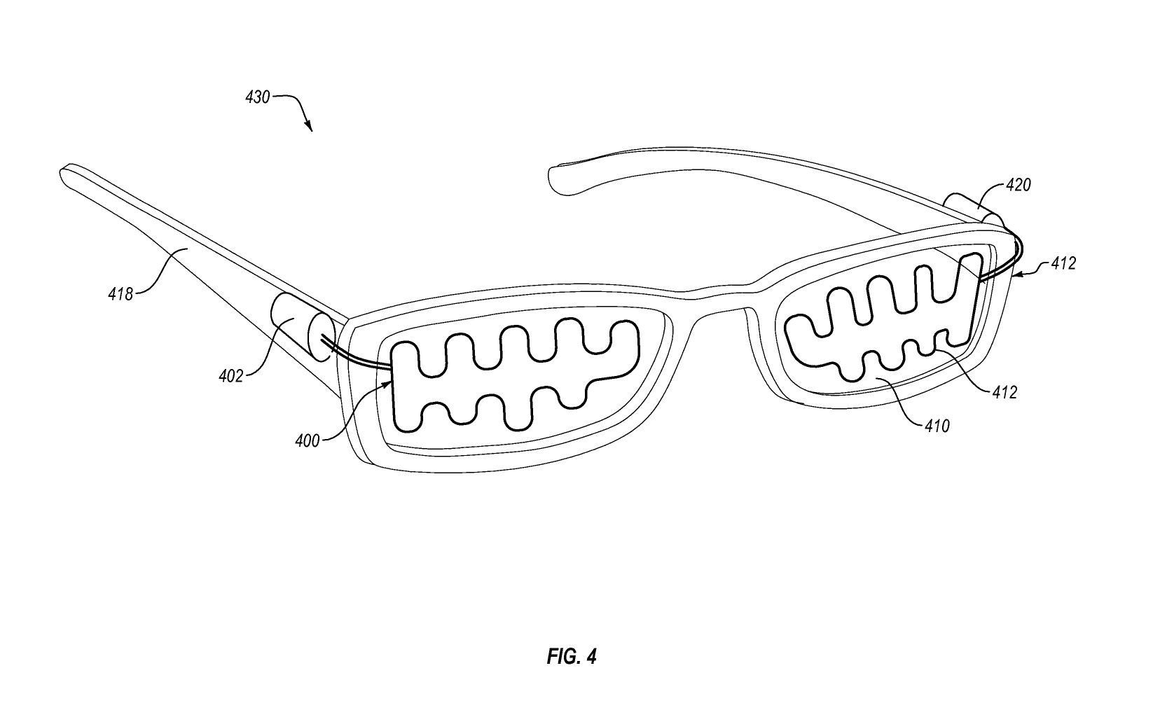

FIG. 4 illustrates another embodiment of an anti-fog system 400 including an anti-fog device 400 attached to a wearable optical device 416. As illustrated, a first power supply 402 can be attached to the wearable optical device 416 at a temple portion 418 (e.g., using a clip and/or other fastener as described above). As shown, the anti-fog device 400 can include a translucent carrier 410 that holds a conductive element 408 (which, in this embodiment, includes a peripheral portion 414 and an inner portion 412) and that allows the anti-fog device 400 to be placed against a viewing portion of the wearable optical device 416.

The illustrated embodiment also includes a second power supply 420, which can be attached to the wearable optical device 416 as the first power supply 402 (e.g., via clip, adhesive, etc.). As shown, the anti-fog device 400 can be configured with two separate and independent circuits providing anti-fogging functionality to separate sections of the wearable optical device 416. Such a configuration can beneficially provide separate and independent control of different sections of the wearable optical device 416 according to user needs and preferences. For example, the first power supply 402 can be configured to operate upon a first detected threshold (e.g., temperature level, humidity level, light transmission level, etc.), while the second power supply can be configured to operate upon a second detected threshold. Additionally, or alternatively, a user may selectively actuate one power supply or the other at different times according to need.

Although the illustrated embodiment shows the separate circuits of the anti-fog device as being positioned side to side (right and left), it will be appreciated that the separate and independent circuits can be arranged in any fashion to provide separately and independently controlled sections of the anti-fog device 400 (e.g., separate horizontal sections, separate concentric sections, etc.).

FIG. 4 also illustrates that certain embodiments can be configured to match the viewing portion of a wearable optical device, even in situations where the viewing portion and/or wearable optical device are irregularly shaped and/or include separate sections. For example, more than one anti-fog device or an anti-fog device having separate sections may be configured to match the size and shape of a desired wearable optical device in order to provide the desired coverage and desired anti-fog effect to the wearable optical device. In such embodiments, the separate sections of the anti-fog device may form separate and independent circuits with separate power supplies (as illustrated by anti-fog device 408, first power supply 402, and second power supply 420), or may alternatively form a single circuit and/or be connected to a single power supply.

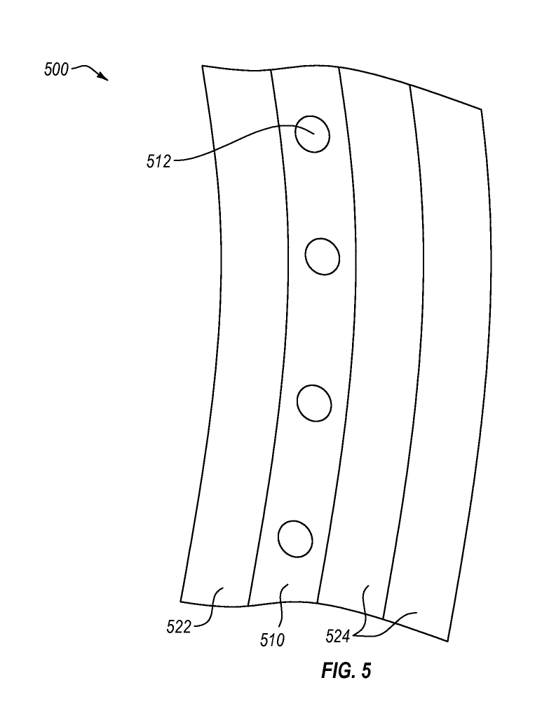

FIG. 5 illustrates a cross-sectional view of an anti-fog device 500. As shown, the anti-fog device 500 can include a conductive element (with the illustrated embodiment showing a section of an inner portion 512 of the conductive element) embedded within a translucent carrier 510. The anti-fog device 500 can also include an adhesive layer 522 disposed on one side of the translucent carrier 510. The adhesive layer 522 can be configured to allow the anti-fog device 500 to be attached to the surface of a wearable optical device or portion thereof.

As illustrated, the anti-fog device 500 can also include one or more additional layers 524. The one or more additional layers 524 may be disposed on a side of the translucent carrier 510 opposite the adhesive layer 522. In other embodiments, the one or more additional layers 524 may be disposed between the translucent carrier 510 and the adhesive layer 522. The one or more additional layers 524 can be formed of plastic, glass, or other translucent material providing additional desired properties to the anti-fog device 500. For example, the one or more additional layers 524 can be configured to provide scratch and/or shatter resistance, to reduce glare and/or polarize light, to be tinted, colored, or shaded to limit the type and/or amount of light passing through, to provide ultra-violet light resistance, or to provide other desired properties. In some embodiments, the one or more additional layers 524 may be configured to provide these properties in addition to such properties provided by the translucent carrier 510.

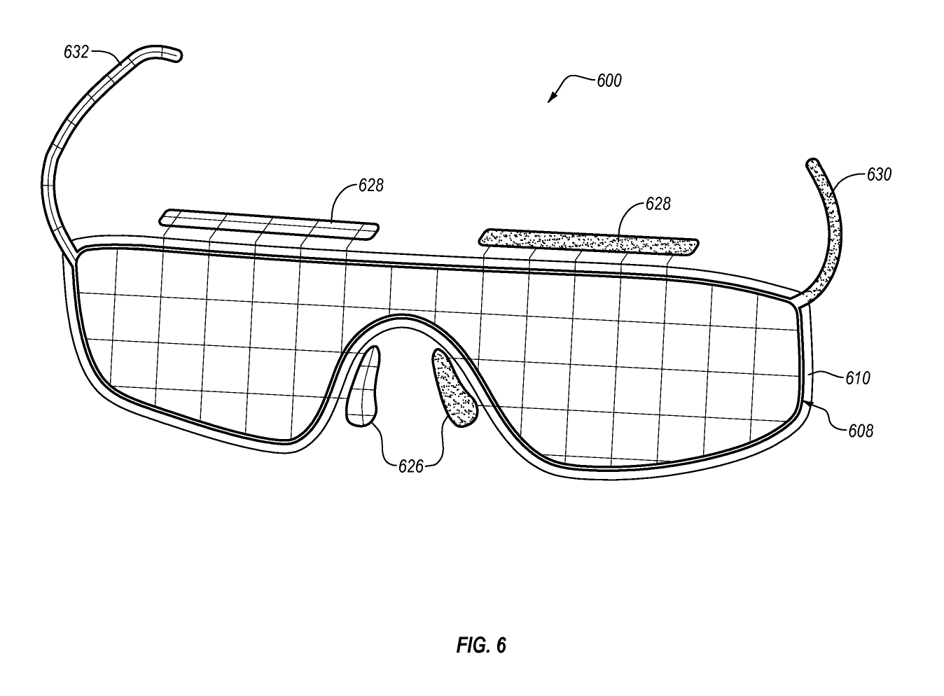

FIG. 6 illustrates an embodiment of an anti-fog device 600 having a conductive element 608 coupled with a translucent carrier 610. As shown, the anti-fog device 600 can include one or more thermal contacts 626, 628, 630, 632. For example, the anti-fog device 600 can include one or more bridge thermal contacts 626, one or more brow thermal contacts 628, one or more temple thermal contacts 630, and one or more ear thermal contacts 632. As shown, the one or more thermal contacts 626, 628, 630, 632 can be conductively coupled to the conductive element 608 so as to enable the transmission of heat from the thermal contacts 626, 628, 630, 632 to the conductive element 608. For example, the bridge thermal contacts 626 can be configured to contact a user's nose or an area close to the user's nose when a wearable optical device incorporating the anti-fog device 600 is donned by the user. Heat generated by the user at and/or near the bridge thermal contacts 626 (e.g., ambient body heat near the user's nose and/or friction generated heat as a result of movement near the bridge thermal contacts 626) can be conducted from the bridge thermal contacts toward and into the conductive element 608.

Similarly, the brow thermal contacts 628 may be configured to contact a user's brow or nearby area, the temple thermal contact 630 may be configured to contact a user's temple or nearby area, and the ear thermal contact 632 may be configured to contact a user's ear or nearby area when a wearable optical device incorporating the anti-fog device 600 is donned by the user. Heat generated by the user at these areas (e.g., ambient body heat and/or friction generated heat) can be conducted from the thermal contacts toward and into the conductive element 608.

As illustrated, the anti-fog device 600 can omit a power supply. In other embodiments, an anti-fog device can include a power supply and one or more thermal contacts. For example, some embodiments may be configured to provide anti-fog functionality through resistive heat generation using a power supply and through thermal transmission of ambient and/or generated heat.

As illustrated, the thermal contacts may be formed as solid components (e.g., as with temple thermal contact 630) or as carrier components extending the wiring of the conductive element 608 to the thermal contact interior (e.g., as with ear thermal contact 632). Where a thermal contact is formed as a solid component, it may be formed of a thermally conductive material, such as aluminum, copper, diamond, gold, molybdenum, tungsten, aluminum nitride, beryllium oxide, silicon carbine, other thermally conductive metals or compounds, or combinations thereof. Where a thermal contact is formed as a carrier component, it may be formed of a film or plastic material (e.g., formed of the same or similar material as the translucent carrier 610) with embedded or attached wiring (e.g., formed of the same or similar material as the wiring of the conductive element 608). Other thermal contacts may include a combination of solid components and carrier components.

The anti-fog device 600 may be attached to a wearable optical device (such as the wearable optical device 216 illustrated in FIG. 2). The thermal contacts can be configured to be attachable to a corresponding section of the wearable optical device so as to be oriented in a position to receive and conduct heat. For example, the bridge thermal contacts 626 can be configured to attach to a nose bridge and/or nose pads of a wearable optical device, the temple thermal contact 630 and the ear thermal contact 632 can be configured to attach to corresponding ears of the wearable optical device, and the brow thermal contacts 628 can be configured to be disposed between the user's brow and the frame of the wearable optical device (e.g., by wrapping up and around the upper portion of the frame). In some embodiments, one or more of the thermal contacts 626, 628, 630, 632 can include an adhesive surface to enable positioning and attachment on the wearable optical device. Additionally, or alternatively, one or more of the thermal contacts 626, 628, 630, 632 can include a clip, pin, hook and loop fastener component, clamp, and/or other fastener.

Other embodiments may include one or more thermal contacts configured to be positioned at other sections of a wearable optical device. For example, an anti-fog device may include a thermal contact configured to be positioned at or near a lower or side portion of a frame of a wearable optical device, or to be positioned at or near other sections of the wearable optical device contacting a user when donned by the user.

One or more embodiments of the present disclosure may also be included in an anti-fog kit. For example, an anti-fog kit can include an anti-fog device, a wearable optical device, and an adhesive for custom attaching of the anti-fog device to the wearable optical device. Custom attaching of the anti-fog device to the wearable optical device can allow a user to position, orient, and/or size the anti-fog device as desired to create a custom wearable optical device having anti-fog functionality. In some embodiments an anti-fog kit can also include a cutting instrument for cutting the optical device to a desired size and shape, one or more batteries for providing power to the power supply, and/or one or more fasteners for attaching the power supply to the wearable optical device (e.g., clips, hook and loop fasteners, etc.).

The terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition.

In addition, unless expressly described otherwise, all stated amounts (e.g., angle measurements, dimension measurements, etc.) are to be interpreted as being “approximately,” “about,” and/or “substantially” the stated amount, regardless of whether the terms “approximately,” “about,” and/or “substantially” are expressly stated in relation to the stated amount(s).

Further, elements described in relation to any embodiment depicted and/or described herein may be combinable with elements described in relation to any other embodiment depicted and/or described herein. For example, any element described in relation to an embodiment depicted in FIGS. 1 through 5 may be combinable with an embodiment described in relation to an embodiment depicted in FIG. 6.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. A modular anti-fog device, comprising:
a translucent carrier formed of a translucent material, the translucent carrier having a first side and a second side, the first side having an adhering surface;
a conductive element formed into a pattern that is positioned within or against the translucent carrier, the conductive element configured to provide heat as a result of the passage of electric current through the conductive element; and
a power supply electrically coupled to the conductive element and configured to provide the conductive element with electric current;
wherein the translucent carrier is configured to be attachable to a wearable optical device by positioning the adhering surface of the translucent carrier against at least a portion of the wearable optical device, thereby providing the wearable optical device with anti-fog functionality or enhanced anti-fog functionality.

2. The device of claim 1, wherein the conductive element is positioned against the first side of the translucent carrier.

3. The device of claim 1, wherein the conductive element is formed in a grid configuration.

4. The device of claim 1, wherein the conductive element is formed having a peripheral portion formed from metal wiring bounding an inner portion formed from metal wiring, the peripheral portion being formed from wiring having a wider diameter than the wiring of the inner portion.

5. The device of claim 1, wherein the conductive element is formed having a peripheral portion formed from metal wiring bounding an inner portion formed from metal wiring, the wiring of the peripheral portion having a lower resistance than the wiring of the inner portion.

6. The device of claim 1, wherein the adhering surface is configured as an adhesive surface.

7. The device of claim 1, wherein the adhering surface is configured to enable adherence to a viewing portion of a wearable optical device through static forces.

8. The device of claim 1, wherein the translucent carrier is formed as a flexible film material.

9. The device of claim 1, further comprising one or more additional layers, at least one of the translucent carrier or one or more additional layers being configured to provide a property selected from the group consisting of scratch-resistance, shatter-resistance, glare reduction, ultra-violet light resistance, and tinting.

10. The device of claim 1, wherein the power supply includes an attachment configured to enable detachable connection of the power supply to a portion of the wearable optical device.

11. The device of claim 1, further comprising a sensor for detecting an ambient condition, the sensor being associated with the power supply so as to actuate or adjust the power supply upon detecting a pre-determined level of the ambient condition.

12. The device of claim 1, further comprising a thermal contact configured to be attachable to a portion of the wearable optical device that is in contact with a user when the wearable optical device is donned by the user, the thermal contact being coupled to the conductive element and configured to receive heat from the user and provide heat to the conductive element.

13. The device of claim 12, wherein the thermal contact is configured as a bridge thermal contact, brow thermal contact, temple thermal contact, or ear thermal contact.

14. The device of claim 12, wherein the thermal contact is configured to receive heat from a user's nose, brow, temple, or ears and conduct the heat to the conductive element.

15. A modular anti-fog device, comprising:
a translucent carrier formed of a translucent material, the translucent carrier having a first side and a second side, the first side having an adhering surface;
a conductive element formed into a pattern that is positioned within or against the translucent carrier, the conductive element configured to receive heat and conduct the heat to an area spanned by the conductive element; and
a thermal contact configured to be attachable to a portion of a wearable optical device that is in contact with a user when the wearable optical device is donned by the user, the thermal contact being coupled to the conductive element and configured to receive heat from the user and provide heat to the conductive element;
wherein the translucent carrier is configured to be attachable to a wearable optical device by positioning the adhering surface of the translucent carrier against at least a portion of the wearable optical device, thereby providing the wearable optical device with anti-fog functionality or enhanced anti-fog functionality.

16. The device of claim 15, wherein the thermal contact is configured as a bridge thermal contact, brow thermal contact, temple thermal contact, or ear thermal contact.

17. The device of claim 15, wherein the thermal contact is configured to receive heat from a user's nose, brow, temple, or ears and conduct the heat to the conductive element.

18. A wearable optical device configured with anti-fog functionality, comprising:
an anti-fog device, including:
a translucent carrier formed of a translucent material, the translucent carrier having a first side and a second side, the first side having an adhering surface;
a conductive element formed into a pattern that is positioned within or against the translucent carrier, the conductive element configured to provide heat as a result of the passage of electric current through the conductive element; and
a power supply electrically coupled to the conductive element and configured to provide the conductive element with electric current; and
a wearable optical device, wherein the translucent carrier is configured to be attachable to the wearable optical device by positioning the adhering surface of the translucent carrier against at least a portion of the wearable optical device, thereby providing the wearable optical device with anti-fog functionality or enhanced anti-fog functionality.

19. The optical device of claim 18, further comprising a thermal contact configured to be attachable to a portion of the wearable optical device that is in contact with a user when the wearable optical device is donned by the user, the thermal contact being coupled to the conductive element and configured to receive heat from the user and provide heat to the conductive element.

20. The optical device of claim 18, wherein the adhering surface is configured as an adhesive surface.
